(19) United States
(12) Patent Application Publication
(10) Pub. No.: US 2010/0130519 A1
(43) Pub. Date: May 27, 2010

(54) COMBINATION THERAPY COMPRISING AZD2171 AND AZD6244 OR MEK-INHIBITOR II

(76) Inventor: Stephen Robert Wedge, Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 12/595,746

(22) PCT Filed: Apr. 11, 2008
(86) PCT No.: PCT/GB08/01266

§ 371 Date: Jan. 4, 2010

(30) Foreign Application Priority Data
Apr. 13, 2007 (GB) 0707145.9
Dec. 5, 2007 (GB) 0723721.7

(51) Int. Cl. A61K 31/517; A61P 35/00

(52) U.S. Cl. 514/266.2

(57) ABSTRACT

The present invention relates to a method for the production of an antiangiogenic and/or vascular permeability reducing effect in a warm-blooded animal such as a human which is optionally being treated with ionising radiation, particularly a method for the treatment of a cancer, particularly a cancer involving a solid tumour, which comprises the administration of AZD2171 in combination with AZD6244 or MEK Inhibitor II; to a pharmaceutical composition comprising AZD2171 and AZD6244 or AZD2171 and MEK Inhibitor II; to a combination product comprising AZD2171 and AZD6244 or AZD2171 and MEK Inhibitor II for use in a method of treatment of a human or animal body by therapy; to a kit comprising AZD2171 and AZD6244 or AZD2171 and MEK Inhibitor II; to the use of AZD2171 and AZD6244 or AZD2171 and MEK Inhibitor II in the manufacture of a medicament for use in the production of an antiangiogenic and/or vascular permeability reducing effect in a warm-blooded animal such as a human which is optionally being treated with ionising radiation.

The present invention relates to a method for the production of an antiangiogenic and/or vascular permeability reducing effect in a warm-blooded animal such as a human which is optionally being treated with ionising radiation, particularly a method for the treatment of a cancer, particularly a cancer involving a solid tumour, which comprises the administration of AZD2171 in combination with AZD6244 or MEK Inhibitor II; to a pharmaceutical composition comprising AZD2171 and AZD6244 or AZD2171 and MEK Inhibitor II; to a combination product comprising AZD2171 and AZD6244 or AZD2171 and MEK Inhibitor II for use in a method of treatment of a human or animal body by therapy; to a kit comprising AZD2171 and AZD6244 or AZD2171 and MEK Inhibitor II; to the use of AZD2171 and AZD6244 or AZD2171 and MEK Inhibitor II in the manufacture of a medicament for use in the production of an antiangiogenic and/or vascular permeability reducing effect in a warm-blooded animal such as a human which is optionally being treated with ionising radiation.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Alteration of vascular permeability is thought to play a role in both normal and pathological physiological processes (Cullinan-Bove et al, 1993, Endocrinology 133: 829-837; Senger et al, 1993, Cancer and Metastasis Reviews, 12: 303-324). Several polypeptides with in vitro endothelial cell growth promoting activity have been identified including, acidic and basic fibroblast growth factors (aFGF & bFGF) and vascular endothelial growth factor (VEGF). By virtue of the restricted expression of its receptors, the growth factor activity of VEGF, in contrast to that of the FGFs, is relatively specific towards endothelial cells. Recent evidence indicates that VEGF is an important stimulator of both normal and pathological angiogenesis (Jakeman et al, 1993, Endocrinology, 133: 848-859; Kolch et al, 1995, Breast Cancer Research and Treatment, 36:139-155) and vascular permeability (Connolly et al, 1989, J. Biol. Chem. 264: 20017-20024). Antagonism of VEGF action by sequestration of VEGF with antibody can result in inhibition of tumour growth (Kim et al, 1993, Nature 362: 841-844).

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fms-like tyrosine kinase receptor, Flt-1 (also referred to as VEGFR-1), the kinase insert domain-containing receptor, KDR (also referred to as VEGFR-2 or Flk-1), and another fms-like tyrosine kinase receptor, Flt-4 (also referred to as VEGFR-3). Two of these related RTKs, Flt-1 and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

VEGF is a key stimulus for vasculogenesis and angiogenesis. This cytokine induces a vascular sprouting phenotype by inducing endothelial cell proliferation, protease expression and migration, and subsequent organisation of cells to form a capillary tube (Keck, P. J., Hauser, S. D., Krivi, G., Sanzo, K., Warren, T., Feder, J., and Connolly, D. T., Science (Washington D.C.), 246: 1309-1312, 1989; Lamoreaux, W. J., Fitzgerald, M. E., Reiner, A., Hasty, K. A., and Charles, S. T., Microvasc. Res., 55: 29-42, 1998; Pepper, M. S., Montesano, R., Mandroita, S. J., Orci, L. and Vassalli, J. D., Enzyme Protein, 49: 138-162, 1996.). In addition, VEGF induces significant vascular permeability (Dvorak, H. F., Detmar, M., Claffey, K. P., Nagy, J. A., van de Water, L., and Senger, D. R., (Int. Arch. Allergy Immunol., 107: 233-235, 1995; Bates, D. O., Heald, R. I., Curry, F. E. and Williams, B. J. Physiol. (Lond.), 533: 263-272, 2001), promoting formation of a hyper-permeable, immature vascular network which is characteristic of pathological angiogenesis.

It has been shown that activation of KDR alone is sufficient to promote all of the major phenotypic responses to VEGF, including endothelial cell proliferation, migration, and survival, and the induction of vascular permeability (Meyer, M., Clauss, M., Lepple-Wienhues, A., Waltenberger, J., Augustin, H. G., Ziche, M., Lanz, C., Buttner, M., Rziha, H-J., and Dehio, C., EMBO J., 18: 363-374, 1999; Zeng, H., Sanyal, S, and Mukhopadhyay, D., J. Biol. Chem., 276: 32714-32719, 2001; Gille, H., Kowalski, J., Li, B., LeCouter, J., Moffat, B, Zioncheck, T. F., Pelletier, N. and Ferrara, N., J. Biol. Chem., 276: 3222-3230, 2001).

Quinazoline derivatives which are inhibitors of VEGF receptor tyrosine kinase are described in International Patent Application Publication No. WO 00/47212. AZD2171 is described in WO 00/47212 and is Example 240 therein. AZD2171 is 4-(4-fluoro-2-methyl-1H-indol-5-yloxy)-6-methoxy-7-(3-(pyrrolidin-1-yl)propoxy)quinazoline:

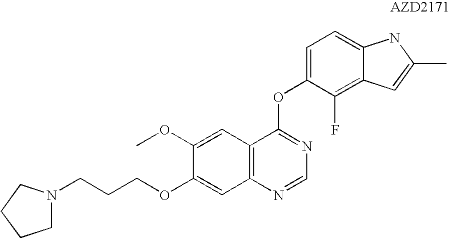

AZD2171 shows excellent activity in the in vitro (a) enzyme and (b) HUVEC assays that are described in WO 00/47212 (pages 80-83). The AZD2171 IC₅₀ values for inhibition of isolated KDR (VEGFR-2), Flt-1 (VEGFR-1) and Flt-4 (VEGFR-3) tyrosine kinase activities in the enzyme assay were <2 nM, 5±2 nM and <3 nM respectively. AZD2171 inhibits VEGF-stimulated endothelial cell proliferation potently (IC₅₀ value of 0.4±0.2 nM in the HUVEC assay), but does not inhibit basal endothelial cell proliferation appreciably at a >1250 fold greater concentration (IC₅₀ value is >500 nM). The growth of a Calu-6 tumour xenograft in the in vivo solid tumour model described in WO 00/47212 (page 83) was inhibited by 49%**, 69%*** and 91%*** following 28 days of once-daily oral treatment with 1.5, 3 and 6 mg/kg/day AZD2171 respectively (P**<0.01, P***<0.0001; one-tailed t test). AZD2171 has been shown to elicit broad-spectrum anti-tumour activity in a range of models following once-daily oral administration, (Wedge et al., 2005, Cancer Research 65: 4389-4440).

AZD2171, as well as producing an antiangiogenic and/or vascular permeability reducing effect by virtue of inhibiting KDR, can have an additional direct antiproliferative effect on tumour cells mediated by inhibition of stem cell factor receptor tyrosine kinase (SCF RTK, commonly known as c-Kit). We have found that AZD2171 inhibits c-Kit and it is expected that AZD2171 will inhibit mutated and wild-type c-Kit. c-Kit and its ligand SCF have been found in numerous solid and haematological malignancies, including gastrointestinal stromal tumours, primary brain tumours such as glioblastoma, glioma and medulloblastoma, small cell lung cancer (SCLC), malignant mesothelioma, tumours of the testis such as seminoma and testicular teratocarcinoma, tumours of the ovary such as dysgerminoma and gonadoblastoma, chronic myelogenous leukaemia (CML), acute myelogenous leukaemia (AML) and mastocytosis (see for example Jnl. Clin. Oncol., 2004, 22, 4514-4522). c-Kit has also been found in hepatocellular carcinoma, (Am J Clin Pathol. 2005 July; 124(1):31-6), and colorectal carcinoma, (Case Reports Tumour Biol. 1993; 14(5):295-302). c-Kit is an important signal transduction inhibitor in certain cancers such as gastrointestinal tumours (GIST), (Bumming et al, 2003 Br J Cancer 89, 460-464), small cell lung cancer (SCLC), (Pott et. al., 2003, Annals of Oncology 14: 894-879), and chronic myelogenous leukaemia (CML), (Goselink et a1.1992, Blood 80, 750-757 and Muroi et al, 1995, Leuk Lymphoma 16, 297-305). c-Kit is also an important signal transduction inhibitor in soft tissue sarcomas like leiomyosarcoma.

In WO 00/47212 it is stated that compounds of the invention: “may be applied as a sole therapy or may involve, in addition to a compound of the invention, one or more other substances and/or treatments. Such conjoint treatment may be achieved by way of the simultaneous, sequential or separate administration of the individual components of the treatment.”

WO 00/47212 then goes on to describe examples of such conjoint treatment including surgery, radiotherapy and various types of chemotherapeutic agent.

Nowhere in WO 00/47212 does it suggest the combination of a compound of the invention and a MEK inhibitor.

Nowhere in WO 00/47212 does it suggest the combination of a compound of the invention and AZD6244 or MEK Inhibitor II for the treatment of any disease state including cancer.

Nowhere in WO 00/47212 is the specific combination of AZD2171 and AZD6244 suggested.

Nowhere in WO 00/47212 is the specific combination of AZD2171 and MEK Inhibitor II suggested.

AZD2171 maleate salt is described in WO 05/061488. In WO 05/061488 it states that “AZD2171 maleate salt is an antiangiogenic and/or vascular permeability reducing agent and may be applied as a sole therapy or may involve, in addition to AZD2171 maleate, one or more other substances and/or treatments. Such conjoint treatment may be achieved by way of the simultaneous, sequential or separate administration of the individual components of the treatment.”

WO 05/016488 then goes on to describe examples of such conjoint treatment including surgery, radiotherapy and various types of chemotherapeutic agent.

Nowhere in WO 05/061488 does it suggest the combination of a compound of the invention and a MEK inhibitor.

Nowhere in WO 05/061488 does it suggest the combination of a compound of the invention and AZD6244 or MEK Inhibitor II for the treatment of any disease state including cancer.

Nowhere in WO 05/061488 is the specific combination of AZD2171 and AZD6244 suggested.

Nowhere in WO 05/061488 is the specific combination of AZD2171 and MEK Inhibitor II suggested.

Nowhere in WO 00/47212 and WO 05/061488 does it state that use of any compounds of the inventions therein with other treatments will produce surprisingly beneficial effects.

Unexpectedly and surprisingly we have now found that the particular compound AZD2171 used in combination with the MEK inhibitor AZD6244, produces significantly better effects than any one of AZD2171 and AZD6244 used alone. In particular, AZD2171 used in combination with AZD6244 produces significantly better effects on solid tumours than any one of AZD2171 and AZD6244 used alone.

Unexpectedly and surprisingly we have now found that the particular compound AZD2171 used in combination with the MEK inhibitor MEK Inhibitor II, produces significantly better effects than any one of AZD2171 and MEK Inhibitor II used alone. In particular, AZD2171 used in combination with MEK Inhibitor II produces significantly better effects on solid tumours than any one of AZD2171 and MEK Inhibitor II used alone.

In recent years it has been discovered that a cell may become cancerous by virtue of the transformation of a portion of its DNA into an oncogene i.e. a gene which, on activation, leads to the formation of malignant tumour cells (Bradshaw, Mutagenesis, 1986, 1, 91). One of the key attributes of malignant cells is the ability to migrate and invade and migrate surrounding tissues leading to host tissue destruction and the formation of secondary metastatic lesions. To achieve this tumour cells must acquire a motile and invasive phenotype as a result of the oncogenic activation of a variety of signalling pathway components. Oncogenes give rise to the production of peptides which are receptors for growth factors. Activation of the growth factor receptor complex subsequently leads to an increase in cell proliferation, motility and invasion. Oncogenes often encode abnormal versions of signal pathway components, such as receptor tyrosine kinases, serine-threonine kinases, or downstream signaling molecules such as the ras genes. The ras genes code for closely related small guanine nucleotide binding proteins which hydrolyse bound guanosine triphosphate (GTP) to guanosine diphosphate (GDP). Ras proteins are active in promoting cell growth, transformation and invasion when they are bound to GTP and inactive when they are bound to GDP. Transforming mutants of p21ras are defective in their GTPase activity and hence remain in the active GTP bound state. The ras oncogene is known to play an integral role in certain cancers and has been found to contribute to the formation of over 20% of all cases of human cancer.

When activated by ligand such as a growth factor, cell surface receptors which are coupled to the mitogenic response can initiate a chain of reactions which leads to the activation of guanine nucleotide exchange activity on ras proteins. When ras protein is in its active GTP-bound state, a number of other proteins interact directly with ras at the plasma membrane resulting in signal transmission through several distinct pathways. The best characterised effector protein is the product of the raf proto-oncogene. The interaction of raf and ras is a key regulatory step in the control of cell proliferation. Ras-mediated activation of the raf serine-threonine kinase in turn activates the dual-specificity kinase MEK (MEK1 and MEK2), which is the immediate upstream activator of mitogen activated protein kinase (MAPKs known as extracellular signal regulated protein kinases or ERK1 and ERK2). To date, no substrates of MEK other than MAPK have been identified, though recent reports indicate that MEK may also be activated by other upstream signal proteins such as MEKK1 and Cot/Tpl-2. Activated MAPK translocates and accumulates in the nucleus, where it can phosphorylate and activate transcription factors such as Elk-1 and Sap 1a, leading to the enhanced expression of genes such as c-fos. In addition, activated MAPK also phosphorylates other kinases eg p90RSK and cytoskeletal proteins.

The ras-dependent raf-MEK-MAPK cascade is one of the key signalling pathways responsible for conveying both mitogenic, survival and invasive signals from cell surface to the nucleus resulting in changes in gene expression and cell fate. Transforming mutants of p21ras are constitutively active, resulting in raf, MEK and MAPK activity and cell transformation. Transforming mutants of BRAF that result in constitutive and elevated kinase activity have been found in approximately 7% of human cancers and result in activation of MEK and MAPK. Inhibition of MEK activity using either antisense raf, a dominant negative MEK mutant or the selective inhibitor PD098059 has been shown to block the growth and morphological transformation of ras-transformed fibroblasts, cell motility and invasion.

The mechanism of activation of raf, MEK and MAPK is through phosphorylation on specific serine, threonine or tyrosine residues. Activated raf and other kinases phosphorylate MEK1 on S218 and 5222 and MEK2 on S222 and S226. This results in MEK activation and subsequent phosphorylation and activation of ERK1 on T190 and Y192 and ERK2 on T183 and Y185 by the dual specificity kinases MEK1 and MEK2. Whilst MEK can be activated by a number of protein kinases, and active MAPKs phosphorylate and activate a number of substrate proteins including transcription factors, other protein kinases and cytosolic proteins, some of which are implicated in the invasive process, MEKs appear specific and sole activators of MAPKs and could act as a focal point for cross-cascade regulation. MEK1 and MEK2 isoforms show unusual specificity and also contain a proline-rich insert between catalytic subdomains IX and X which is not present in any of the other known MEK family members. These differences between MEK and other protein kinases, together with the known role of MEK (MEK 1, MEK 2) and, more recently MEK 5, in proliferative and invasive signalling suggest it may be possible to discover and employ selective MEK inhibitors as therapeutic agents for use in proliferative and invasive disease.

Accordingly, it has been recognised that an inhibitor of the MAPK kinase pathway should be of value both as an anti-proliferative and anti-invasive agent for use in the containment and/or treatment of solid tumour disease.

MEK inhibitors are described in International Patent Application Publication No. WO 03/077914. AZD6244 is described in WO 03/077914 and is Example 10 therein. AZD6244 is 6-(4-Bromo-2-chloro-phenylamino)-7-fluoro-3-methyl-3H-benzoimidazole-5-carboxylic acid (2-hydroxy-ethoxy)-amide:

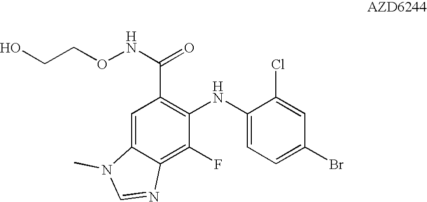

MEK Inhibitor II is 2-(2-fluoro-4-iodophenylamino)-N-(2-hydroxyethoxy)-1,5-dimethyl-6-oxo-1,6-dihydropyridine-3-carboxamide:

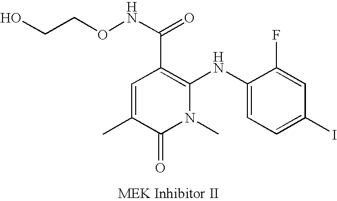

Anti-cancer effects of a method of treatment of the present invention include, but are not limited to, anti-tumour effects, the response rate, the time to disease progression and the survival rate. Anti-tumour effects of a method of treatment of the present invention include but are not limited to, inhibition of tumour growth, tumour growth delay, regression of tumour, shrinkage of tumour, increased time to regrowth of tumour on cessation of treatment, slowing of disease progression. It is expected that when a method of treatment of the present invention is administered to a warm-blooded animal such as a human, in need of treatment for cancer, said method of treatment will produce an effect, as measured by, for example, one or more of: the extent of the anti-tumour effect, the response rate, the time to disease progression and the survival rate. Anti-cancer effects include prophylactic treatment as well as treatment of existing disease.

According to the present invention there is provided a method for the production of an antiangiogenic and/or vascular permeability reducing effect in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

According to a further aspect of the present invention there is provided a method for the treatment of a cancer in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

According to a further aspect of the present invention there is provided a method for the treatment of a cancer involving a solid tumour in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

According to a further aspect of the present invention there is provided a method for the treatment of non-small cell lung cancer (NSCLC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

According to a further aspect of the present invention there is provided a method for the treatment of colorectal cancer (CRC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

According to a further aspect of the present invention there is provided a method for the treatment of cancer of the pancreas in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

According to a further aspect of the present invention there is provided a method for the treatment of malignant melanoma in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

According to a further aspect of the present invention there is provided a method for the production of an antiangiogenic and/or vascular permeability reducing effect in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof; wherein AZD2171 and AZD6244 or MEK Inhibitor II may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof; wherein AZD2171 and AZD6244 or MEK Inhibitor II may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof; wherein AZD2171 and AZD6244 or MEK Inhibitor II may each optionally be administered together with a pharmaceutically acceptable excipient or carrier.

According to a further aspect of the present invention there is provided a method for the treatment of non-small cell lung cancer (NSCLC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof; wherein AZD2171 and AZD6244 or MEK Inhibitor II may each optionally be administered together with a pharmaceutically acceptable excipient or carrier.

According to a further aspect of the present invention there is provided a method for the treatment of colorectal cancer (CRC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof; wherein AZD2171 and AZD6244 or MEK Inhibitor II may each optionally be administered together with a pharmaceutically acceptable excipient or carrier.

According to a further aspect of the present invention there is provided a method for the treatment of cancer of the pancreas in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof; wherein AZD2171 and AZD6244 or MEK Inhibitor II may each optionally be administered together with a pharmaceutically acceptable excipient or carrier.

According to a further aspect of the present invention there is provided a method for the treatment of malignant melanoma in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof; wherein AZD2171 and AZD6244 or MEK Inhibitor II may each optionally be administered together with a pharmaceutically acceptable excipient or carrier.

According to a further aspect of the invention there is provided a pharmaceutical composition which comprises AZD2171 or a pharmaceutically acceptable salt thereof, and AZD6244 or a pharmaceutically acceptable salt thereof, in association with a pharmaceutically acceptable excipient or carrier.

According to a further aspect of the invention there is provided a pharmaceutical composition which comprises AZD2171 or a pharmaceutically acceptable salt thereof, and MEK Inhibitor II or a pharmaceutically acceptable salt thereof, in association with a pharmaceutically acceptable excipient or carrier.

According to a further aspect of the present invention there is provided a combination product comprising AZD2171 or a pharmaceutically acceptable salt thereof and AZD6244 or a pharmaceutically acceptable salt thereof, for use in a method of treatment of a human or animal body by therapy.

According to a further aspect of the present invention there is provided a combination product comprising AZD2171 or a pharmaceutically acceptable salt thereof and MEK Inhibitor II or a pharmaceutically acceptable salt thereof, for use in a method of treatment of a human or animal body by therapy.

According to a further aspect of the present invention there is provided a kit comprising AZD2171 or a pharmaceutically acceptable salt thereof, and AZD6244 or a pharmaceutically acceptable salt thereof.

According to a further aspect of the present invention there is provided a kit comprising:

a) AZD2171 or a pharmaceutically acceptable salt thereof in a first unit dosage form;
b) AZD6244 or a pharmaceutically acceptable salt thereof in a second unit dosage form; and
c) container means for containing said first and second dosage forms.

According to a further aspect of the present invention there is provided a kit comprising:

a) AZD2171 or a pharmaceutically acceptable salt thereof, together with a pharmaceutically acceptable excipient or carrier, in a first unit dosage form;
b) AZD6244 or a pharmaceutically acceptable salt thereof together with a pharmaceutically acceptable excipient or carrier, in a second unit dosage form; and
c) container means for containing said first and second dosage forms.

According to a further aspect of the present invention there is provided a kit comprising AZD2171 or a pharmaceutically acceptable salt thereof, and MEK Inhibitor II or a pharmaceutically acceptable salt thereof.

According to a further aspect of the present invention there is provided a kit comprising:

a) AZD2171 or a pharmaceutically acceptable salt thereof in a first unit dosage form;
b) MEK Inhibitor II or a pharmaceutically acceptable salt thereof in a second unit dosage form; and
c) container means for containing said first and second dosage forms.

According to a further aspect of the present invention there is provided a kit comprising:

a) AZD2171 or a pharmaceutically acceptable salt thereof, together with a pharmaceutically acceptable excipient or carrier, in a first unit dosage form;
b) MEK Inhibitor II or a pharmaceutically acceptable salt thereof together with a pharmaceutically acceptable excipient or carrier, in a second unit dosage form; and
c) container means for containing said first and second dosage forms.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an antiangiogenic and/or vascular permeability reducing effect in a warm-blooded animal such as a human.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof in the manufacture of a medicament for use in the production of an anti-cancer effect in a warm-blooded animal such as a human wherein the cancer is non-small cell lung cancer (NSCLC).

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human wherein the cancer is colorectal cancer (CRC).

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human wherein the cancer is pancreatic cancer.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human wherein the cancer is malignant melanoma.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human wherein the tumour is a non-small cell tumour of the lung.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human wherein the tumour is a tumour of the colon or rectum.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human wherein the tumour is a tumour of the pancreas.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human wherein the tumour is a malignant melanoma.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an antiangiogenic and/or vascular permeability reducing effect in a warm-blooded animal such as a human.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human wherein the cancer is non-small cell lung cancer (NSCLC).

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human wherein the cancer is colorectal cancer (CRC).

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human wherein the cancer is pancreatic cancer.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human wherein the cancer is malignant melanoma.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human wherein the tumour is a non-small cell tumour of the lung.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human wherein the tumour is a tumour of the colon or rectum.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human wherein the tumour is a tumour of the pancreas.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human wherein the tumour is a malignant melanoma.

According to a further aspect of the present invention there is provided a combination treatment comprising the administration of an effective amount of AZD2171 or a pharmaceutically acceptable salt thereof, optionally together with a pharmaceutically acceptable excipient or carrier, and the simultaneous, sequential or separate administration of an effective amount of either AZD6244 or a pharmaceutically acceptable salt thereof or MEK Inhibitor II or a pharmaceutically acceptable salt thereof wherein AZD6244 or MEK Inhibitor II may optionally be administered together with a pharmaceutically acceptable excipient or carrier; to a warm-blooded animal such as a human in need of such therapeutic treatment.

Such therapeutic treatment includes an antiangiogenic and/or vascular permeability effect, an anti-cancer effect and an anti-tumour effect.

A combination treatment of the present invention as defined herein may be achieved by way of the simultaneous, sequential or separate administration of the individual components of said treatment. A combination treatment as defined herein may be applied as a sole therapy or may involve surgery or radiotherapy or an additional chemotherapeutic agent in addition to a combination treatment of the invention.

Surgery may comprise the step of partial or complete tumour resection, prior to, during or after the administration of the combination treatment with AZD2171 described herein.

Other chemotherapeutic agents for optional use with a combination treatment of the present invention include those described in WO 00/47212 which is incorporated herein by reference. Such chemotherapy may cover nine main categories of therapeutic agent:

(i) other antiangiogenic agents including vascular targeting agents;

(ii) cytostatic agents;

(iii) biological response modifiers (for example interferon);

(iv) antibodies (for example edrecolomab); and

(v) antiproliferative/antineoplastic drugs and combinations thereof, as used in medical oncology; and other categories of agent are:

(vi) antisense therapies;

(vii) gene therapy approaches; and

(ix) immunotherapy approaches.

Particular examples of chemotherapeutic agents for use with a combination treatment of the present invention are pemetrexed, raltitrexed, etoposide, vinorelbine, paclitaxel, docetaxel, cisplatin, oxaliplatin, carboplatin, gemcitabine, irinotecan (CPT-11), 5-fluorouracil (5-FU, (including capecitabine)), doxorubicin, cyclophosphamide, temozolomide and hydroxyurea. Such combinations are expected to be particularly useful for the treatment of cancer of the lung, head and neck, brain, colon, rectum, esophagus, stomach, cervix, ovary, skin, breast, bladder, prostate, pancreas and including haematological malignancies. Such combinations are expected to be more particularly useful for the treatment of cancer of the pancreas, colorectal cancer, malignant melanoma and non-small cell lung cancer (NSCLC).

The administration of a triple combination of AZD2171, AZD6244 and ionising radiation may produce effects, such as anti-tumour effects, greater than those achieved with any of AZD2171, AZD6244 and ionising radiation used alone, greater than those achieved with the combination of AZD2171 and AZD6244, greater than those achieved with the combination of AZD2171 and ionising radiation, greater than those achieved with the combination of AZD6244 and ionising radiation.

The administration of a triple combination of AZD2171, MEK Inhibitor II and ionising radiation may produce effects, such as anti-tumour effects, greater than those achieved with any of AZD2171, MEK Inhibitor II and ionising radiation used alone, greater than those achieved with the combination of AZD2171 and MEK Inhibitor II, greater than those achieved with the combination of AZD2171 and ionising radiation, greater than those achieved with the combination of MEK Inhibitor II and ionising radiation.

According to the present invention there is provided a method for the production of an antiangiogenic and/or vascular permeability reducing effect in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

According to a further aspect of the present invention there is provided a method for the treatment of a cancer in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

According to a further aspect of the present invention there is provided a method for the treatment of a cancer involving a solid tumour in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

According to a further aspect of the present invention there is provided a method for the treatment of non-small cell lung cancer (NSCLC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

According to a further aspect of the present invention there is provided a method for the treatment of colorectal cancer (CRC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

According to a further aspect of the present invention there is provided a method for the treatment of cancer of the pancreas in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

According to a further aspect of the present invention there is provided a method for the treatment of malignant melanoma in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

According to a further aspect of the present invention there is provided a method for the production of an antiangiogenic and/or vascular permeability reducing effect in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation, wherein AZD2171 and AZD6244 or MEK Inhibitor II may each optionally be administered together with a pharmaceutically acceptable excipient or carrier.

According to a further aspect of the present invention there is provided a method for the treatment of a cancer in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation, wherein AZD2171 and AZD6244 or MEK Inhibitor II may each optionally be administered together with a pharmaceutically acceptable excipient or carrier.

According to a further aspect of the present invention there is provided a method for the treatment of a cancer involving a solid tumour in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation, wherein AZD2171 and AZD6244 or MEK Inhibitor II may each optionally be administered together with a pharmaceutically acceptable excipient or carrier.

According to a further aspect of the present invention there is provided a method for the treatment of non-small cell lung cancer (NSCLC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation, wherein AZD2171 and AZD6244 or MEK Inhibitor II may each optionally be administered together with a pharmaceutically acceptable excipient or carrier.

According to a further aspect of the present invention there is provided a method for the treatment of colorectal cancer (CRC) in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation, wherein AZD2171 and AZD6244 or MEK Inhibitor II may each optionally be administered together with a pharmaceutically acceptable excipient or carrier.

According to a further aspect of the present invention there is provided a method for the treatment of cancer of the pancreas in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation, wherein AZD2171 and AZD6244 or MEK Inhibitor II may each optionally be administered together with a pharmaceutically acceptable excipient or carrier.

According to a further aspect of the present invention there is provided a method for the treatment of malignant melanoma in a warm-blooded animal such as a human, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation, wherein AZD2171 and AZD6244 or MEK Inhibitor II may each optionally be administered together with a pharmaceutically acceptable excipient or carrier.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an antiangiogenic and/or vascular permeability reducing effect in a warm-blooded animal such as a human which is being treated with ionising radiation.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human which is being treated with ionising radiation.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human which is being treated with ionising radiation.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human which is being treated with ionising radiation wherein the cancer is non-small cell lung cancer (NSCLC).

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human which is being treated with ionising radiation wherein the cancer is cancer of the colon or rectum.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human which is being treated with ionising radiation wherein the cancer is pancreatic cancer.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-cancer effect in a warm-blooded animal such as a human which is being treated with ionising radiation wherein the cancer is malignant melanoma.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human which is being treated with ionising radiation wherein the tumour is a non-small cell tumour of the lung.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human which is being treated with ionising radiation wherein the tumour is a tumour of the colon or rectum.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human which is being treated with ionising radiation wherein the tumour is a tumour of the pancreas.

According to a further aspect of the present invention there is provided the use of AZD2171 or a pharmaceutically acceptable salt thereof and either AZD6244 or a pharmaceutically acceptable salt thereof or MEK Inhibitor II or a pharmaceutically acceptable salt thereof, in the manufacture of a medicament for use in the production of an anti-tumour effect in a warm-blooded animal such as a human which is being treated with ionising radiation wherein the tumour is a malignant melanoma.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an antiangiogenic and/or vascular permeability reducing effect in a warm-blooded animal such as a human which is being treated with ionising radiation.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human which is being treated with ionising radiation.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human which is being treated with ionising radiation.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human which is being treated with ionising radiation wherein the cancer is non-small cell lung cancer (NSCLC).

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human which is being treated with ionising radiation wherein the cancer is cancer of the colon or rectum.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human which is being treated with ionising radiation wherein the cancer is pancreatic cancer.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-cancer effect in a warm-blooded animal such as a human which is being treated with ionising radiation wherein the cancer is malignant melanoma.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human which is being treated with ionising radiation wherein the tumour is a non-small cell tumour of the lung.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human which is being treated with ionising radiation wherein the tumour is a tumour of the colon or rectum.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human which is being treated with ionising radiation wherein the tumour is a tumour of the pancreas.

According to a further aspect of the present invention there is provided AZD2171 or a pharmaceutically acceptable salt thereof and either AZD6244 or a pharmaceutically acceptable salt thereof or MEK Inhibitor II or a pharmaceutically acceptable salt thereof, for use in the production of an anti-tumour effect in a warm-blooded animal such as a human which is being treated with ionising radiation wherein the tumour is a malignant melanoma.

According to a further aspect of the present invention there is provided a therapeutic combination treatment comprising the administration of an effective amount of AZD2171 or a pharmaceutically acceptable salt thereof, optionally together with a pharmaceutically acceptable excipient or carrier, and the administration of an effective amount of AZD6244 or a pharmaceutically acceptable salt thereof, optionally together with a pharmaceutically acceptable excipient or carrier and the administration of an effective amount of ionising radiation, to a warm-blooded animal such as a human in need of such therapeutic treatment wherein the AZD2171, AZD6244 and ionising radiation may be administered simultaneously, sequentially or separately and in any order.

According to a further aspect of the present invention there is provided a therapeutic combination treatment comprising the administration of an effective amount of AZD2171 or a pharmaceutically acceptable salt thereof, optionally together with a pharmaceutically acceptable excipient or carrier, and the administration of an effective amount of MEK Inhibitor II or a pharmaceutically acceptable salt thereof, optionally together with a pharmaceutically acceptable excipient or carrier and the administration of an effective amount of ionising radiation, to a warm-blooded animal such as a human in need of such therapeutic treatment wherein the AZD2171, MEK Inhibitor II and ionising radiation may be administered simultaneously, sequentially or separately and in any order.

A warm-blooded animal such as a human which is being treated with ionising radiation means a warm-blooded animal such as a human which is treated with ionising radiation before, after or at the same time as the administration of a medicament or combination treatment comprising AZD2171 and AZD6244 or AZD2171 and MEK Inhibitor II. For example said ionising radiation may be given to said warm-blooded animal such as a human within the period of a week before to a week after the administration of a medicament or combination treatment comprising AZD2171 and AZD6244 or AZD2171 and MEK Inhibitor II. This means that AZD2171, AZD6244 or MEK Inhibitor II and ionising radiation may be administered separately or sequentially in any order, or may be administered simultaneously. The warm-blooded animal may experience the effect of each of AZD2171, AZD6244 or MEK Inhibitor II, and radiation simultaneously. According to one aspect of the present invention the ionising radiation is administered before one of AZD2171 and AZD6244 or after one of AZD2171 and AZD6244.

According to one aspect of the present invention the ionising radiation is administered before both AZD2171 and AZD6244 or after both AZD2171 and AZD6244.

According to one aspect of the present invention the ionising radiation is administered before one of AZD2171 and MEK Inhibitor II or after one of AZD2171 and MEK Inhibitor II.

According to one aspect of the present invention the ionising radiation is administered before both AZD2171 and MEK Inhibitor II or after both AZD2171 and MEK Inhibitor II.

According to one aspect of the present invention AZD2171 is administered to a warm-blooded animal after the animal has been treated with ionising radiation.

According to another aspect of the present invention the effect of a method of treatment of the present invention is expected to be at least equivalent to the addition of the effects of each of the components of said treatment used alone, that is, of each of AZD2171 and AZD6244 used alone or of each of AZD2171, AZD6244 and ionising radiation used alone.

According to another aspect of the present invention the effect of a method of treatment of the present invention is expected to be greater than the addition of the effects of each of the components of said treatment used alone, that is, of each of AZD2171 and AZD6244 used alone or of each of AZD2171, AZD6244 and ionising radiation used alone.

According to another aspect of the present invention the effect of a method of treatment of the present invention is expected to be at least equivalent to the addition of the effects of each of the components of said treatment used alone, that is, of each of AZD2171 and MEK Inhibitor II used alone or of each of AZD2171, MEK Inhibitor II and ionising radiation used alone.

According to another aspect of the present invention the effect of a method of treatment of the present invention is expected to be greater than the addition of the effects of each of the components of said treatment used alone, that is, of each of AZD2171 and MEK Inhibitor II used alone or of each of AZD2171, MEK Inhibitor II and ionising radiation used alone.

According to another aspect of the present invention the effect of a method of treatment of the present invention is expected to be a synergistic effect.

According to the present invention a combination treatment is defined as affording a synergistic effect if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with AZD2171 or AZD6244 or MEK Inhibitor II or ionising radiation alone. Further, the effect of the combination treatment is synergistic if a beneficial effect is obtained in a group of patients that does not respond (or responds poorly) to AZD2171 or AZD6244 or MEK Inhibitor II or ionising radiation alone. In addition, the effect of the combination treatment is defined as affording a synergistic effect if one of the components is dosed at its conventional dose and the other component(s) is/are dosed at a reduced dose and the therapeutic effect, as measured by, for example, the extent of the response, the response rate, the time to disease progression or the survival period, is equivalent to that achievable on dosing conventional amounts of the components of the combination treatment. In particular, synergy is deemed to be present if the conventional dose of AZD2171 or AZD6244 or MEK Inhibitor II or ionising radiation may be reduced without detriment to one or more of the extent of the response, the response rate, the time to disease progression and survival data, in particular without detriment to the duration of the response, but with fewer and/or less troublesome side-effects than those that occur when conventional doses of each component are used.

As stated above the combination treatments of the present invention as defined herein are of interest for their antiangiogenic and/or vascular permeability effects. Angiogenesis and/or an increase in vascular permeability is present in a wide range of disease states including cancer (including leukaemia, multiple myeloma and lymphoma), diabetes, psoriasis, rheumatoid arthritis, Kaposi's sarcoma, haemangioma, acute and chronic nephropathies, atheroma, arterial restenosis, autoimmune diseases, acute inflammation, asthma, lymphoedema, endometriosis, dysfunctional uterine bleeding and ocular diseases with retinal vessel proliferation including age-related macular degeneration.

Combination treatments of the present invention are expected to be particularly useful in the prophylaxis and treatment of diseases such as cancer and Kaposi's sarcoma. In particular such combination treatments of the invention are expected to be useful in the treatment of cancer, for example cancer of the lung, head and neck, brain, colon, rectum, esophagus, stomach, liver, biliary tract, thyroid, kidney, cervix, ovary, uterus, skin, breast, bladder, prostate, pancreas and including haematological malignancies such as leukaemia, multiple myeloma and lymphoma. In particular such combination treatments of the invention are expected to slow advantageously the growth of primary and recurrent solid tumours of, for example, the colon, rectum, pancreas, brain, bladder, ovary, breast, prostate, lungs, liver and skin. Combination treatments of the present invention are expected to slow advantageously the growth of tumours in malignant melanoma, colorectal cancer, pancreatic cancer, hepatocellular cancer and lung cancer including non-small cell lung cancer (NSCLC). Combination treatments of the present invention are expected to slow advantageously the growth of tumours in malignant melanoma, colorectal cancer, pancreatic cancer and lung cancer including non-small cell lung cancer (NSCLC). More particularly such combination treatments of the invention are expected to inhibit any form of cancer associated with VEGF including leukaemia, multiple myeloma and lymphoma and also, for example, to inhibit the growth of those primary and recurrent solid tumours which are associated with VEGF, especially those tumours which are significantly dependent on VEGF for their growth and spread, including for example, certain tumours of the colon, rectum, pancreas, brain, bladder, ovary, breast, prostate, lung, vulva, liver and skin. More especially combination treatments of the present invention are expected to slow advantageously the growth of tumours in malignant melanoma. More especially combination treatments of the present invention are expected to slow advantageously the growth of tumours in non-small cell lung cancer (NSCLC). More especially combination treatments of the present invention are expected to slow advantageously the growth of tumours in colorectal cancer (CRC). More especially combination treatments of the present invention are expected to slow advantageously the growth of tumours in pancreatic cancer. More especially combination treatments of the present invention are expected to slow advantageously the growth of tumours in hepatocellular cancer.

In another aspect of the present invention AZD2171 and AZD6244, or AZD2171 and MEK Inhibitor II, optionally with ionising radiation, are expected to inhibit the growth of those primary and recurrent solid tumours which are associated with VEGF especially those tumours which are significantly dependent on VEGF for their growth and spread.

The compositions described herein may be in a form suitable for oral administration, for example as a tablet or capsule, for nasal administration or administration by inhalation, for example as a powder or solution, for parenteral injection (including intravenous, subcutaneous, intramuscular, intravascular or infusion) for example as a sterile solution, suspension or emulsion, for topical administration for example as an ointment or cream, for rectal administration for example as a suppository or the route of administration may be by direct injection into the tumour or by regional delivery or by local delivery. In other embodiments of the present invention the AZD2171, AZD6244 or MEK Inhibitor II of the combination treatment may be delivered endoscopically, intratracheally, intralesionally, percutaneously, intravenously, subcutaneously, intraperitoneally or intratumourally. Preferably AZD2171 is administered orally. Preferably AZD6244 is administered orally. Preferably MEK Inhibitor II is administered orally. In general the compositions described herein may be prepared in a conventional manner using conventional excipients. The compositions of the present invention are advantageously presented in unit dosage form.

AZD2171 will normally be administered to a warm-blooded animal at a unit dose within the range 1-50 mg per square metre body area of the animal, for example approximately 0.03-1.5 mg/kg in a human. A unit dose in the range, for example, 0.01-1.5 mg/kg, preferably 0.03-0.5 mg/kg is envisaged and this is normally a therapeutically-effective dose. A unit dosage form such as a tablet or capsule will usually contain, for example 1-50 mg of active ingredient. Preferably a daily dose in the range of 0.03-0.5 mg/kg is employed.

AZD6244 will normally be administered to a warm-blooded animal so that a daily dose in the range, for example, 0.1 mg/kg to 75 mg/kg body weight is received, given, if required, in divided doses. AZD6244 may be administered orally such as in a tablet or capsule. AZD6244 may also be administered parenterally. In such cases lower doses will be used. Thus, for example, for intravenous administration, a dose in the range 0.1 mg/kg to 30 mg/kg body weight will generally be used.

MEK Inhibitor II will normally be administered to a warm-blooded animal so that a daily dose in the range, for example, 0.1 mg/kg to 75 mg/kg body weight is received, given, if required, in divided doses. MEK Inhibitor II may be administered orally such as in a tablet or capsule. MEK Inhibitor II may also be administered parenterally. In such cases lower doses will be used. Thus, for example, for intravenous administration, a dose in the range 0.1 mg/kg to 30 mg/kg body weight will generally be used.

In combinations of the invention AZD2171 would normally be combined with AZD6244 or MEK Inhibitor II, however, in some embodiments of the invention a patient treated with AZD6244 could be switched to MEK Inhibitor II or a patient treated with MEK inhibitor II could be switched to AZD6244.

The dosages and schedules may vary according to the particular disease state and the overall condition of the patient. Dosages and schedules may also vary if, in addition to a combination treatment of the present invention, one or more additional chemotherapeutic agents is/are used. Scheduling can be determined by the practitioner who is treating any particular patient.

Radiotherapy may be administered according to the known practices in clinical radiotherapy. The dosages of ionising radiation will be those known for use in clinical radiotherapy. The radiation therapy used will include for example the use of γ-rays, X-rays, and/or the directed delivery of radiation from radioisotopes. Other forms of DNA damaging factors are also included in the present invention such as microwaves and UV-irradiation. For example X-rays may be dosed in daily doses of 1.8-2.0 Gy, 5 days a week for 5-6 weeks. Normally a total fractionated dose will lie in the range 45-60 Gy. Single larger doses, for example 5-10 Gy may be administered as part of a course of radiotherapy. Single doses may be administered intraoperatively. Hyperfractionated radiotherapy may be used whereby small doses of X-rays are administered regularly over a period of time, for example 0.1 Gy per hour over a number of days. Dosage ranges for radioisotopes vary widely, and depend on the half-life of the isotope, the strength and type of radiation emitted, and on the uptake by cells.

The size of the dose of each therapy which is required for the therapeutic or prophylactic treatment of a particular disease state will necessarily be varied depending on the host treated, the route of administration and the severity of the illness being treated. Accordingly the optimum dosage may be determined by the practitioner who is treating any particular patient. For example, it may be necessary or desirable to reduce the above-mentioned doses of the components of the combination treatments in order to reduce toxicity.

The present invention relates to combinations of AZD6244 with AZD2171 or with a salt of AZD2171 and to combinations of MEK Inhibitor II with AZD2171 or with a salt of AZD2171.

Salts of AZD2171 for use in pharmaceutical compositions will be pharmaceutically acceptable salts, but other salts may be useful in the production of AZD2171 and its pharmaceutically acceptable salts. Pharmaceutically acceptable salts may, for example, include acid addition salts. Such acid addition salts include for example salts with inorganic or organic acids affording pharmaceutically acceptable anions such as with hydrogen halides or with sulphuric or phosphoric acid, or with trifluoroacetic, citric or maleic acid. In addition pharmaceutically acceptable salts may be formed with an inorganic or organic base which affords a pharmaceutically acceptable cation. Such salts with inorganic or organic bases include for example an alkali metal salt, such as a sodium or potassium salt and an alkaline earth metal salt such as a calcium or magnesium salt. A preferred salt is AZD2171 maleate which is described in International Patent Application Publication No. WO 05/061488.

Salts of AZD6244 for use in pharmaceutical compositions will be pharmaceutically acceptable salts, but other salts may be useful in the production of AZD6244 and its pharmaceutically acceptable salts. A preferred salt is AZD6244 hydrogen sulphate salt.

Salts of MEK Inhibitor II for use in pharmaceutical compositions will be pharmaceutically acceptable salts, but other salts may be useful in the production of MEK Inhibitor II and its pharmaceutically acceptable salts.

AZD2171 may be synthesised according to the processes described in WO 00/47212, in particular those described in Example 240 of WO 00/47212.

AZD2171 maleate salt may be synthesised according to the processes described in WO 05/061488.

AZD6244 may be synthesised according to the processes described in WO 03/077914, in particular those described in Example 10 of WO 03/077914.

The hydrogen sulphate salt of AZD6244 can be prepared by reacting a slurry of AZD6244 in an organic liquid, such as a C₁₋₆ alkyl ketone, with at least a stoichiometric amount of sulphuric acid and water and thereafter recovering the salt from the resultant solution, such as by cooling the solution mixture to precipitate the salt.

MEK Inhibitor II may be synthesised as follows:

Step A. Preparation of 2-chloro-6-oxo-1,6-dihydro-pyridine-3-carboxylic acid: 2-Chloro-6-oxo-1,6-dihydro-pyridine-3-carboxylic acid was prepared from dichloronicotinic acid (3.00 g, 15.6 mmol, Aldrich) according to the procedure described in U.S. Pat. No. 3,682,932 to yield 1.31 g (48%) of the desired product.

Step B. Preparation of 2-chloro-1-methyl-6-oxo-1,6-dihydro-pyridine-3-carboxylic acid methyl ester: To a solution of 2-chloro-6-oxo-1,6-dihydro-pyridine-3-carboxylic acid (0.644 g, 3.71 mmol) in DMF (20 mL) was added lithium hydride (95%, 0.078 g, 9.28 mmol) and the reaction mixture was stirred for 40 minutes under N₂. Methyl iodide (0.508 mL, 1.16 g, 8.16 mmol) was then added and the reaction mixture was stirred for an additional 45 minutes. The reaction mixture was quenched with 2 M HCl until the pH was 6-7. The reaction mixture was diluted with EtOAc and saturated NaCl and the layers separated. The aqueous layer was back extracted with EtOAc (1×). The combined organic layers were dried (Na₂SO₄) and concentrated under reduced pressure to yield a crude yellow solid. HPLC analysis showed two products in a 4:1 ratio that were separated by flash column chromatography (methylene chloride/EtOAc, 15:1 to 10:1) to give 0.466 g (62%) pure desired product as a white crystalline solid.

Step C. Preparation of methyl 5-bromo-2-chloro-1-methyl-6-oxo-1,6-dihydropyridine-3-carboxylate: To a solution of methyl 2-chloro-1-methyl-6-oxo-1,6-dihydropyridine-3-carboxylate (0.100 g, 0.496 mmol) in DMF (5 mL) was added N-bromosuccinimide (0.177 g, 0.992 mmol) and the reaction mixture was stirred for 4 hours at room temperature under N₂. The reaction mixture was quenched with saturated sodium bisulfite and then diluted with EtOAc and H₂O and the layers separated. The aqueous layer was back extracted with EtOAc (2×). The combined organic layers were dried (Na₂SO₄) and concentrated under reduced pressure to yield a yellow solid in quantitative yield.

Step D. Preparation of methyl 2-chloro-1,5-dimethyl-6-oxo-1,6-dihydropyridine-3-carboxylate: To a suspension of methyl 5-bromo-2-chloro-1-methyl-6-oxo-1,6-dihydropyridine-3-carboxylate (0.400 g, 1.43 mmol) and 1,1′-bis(diphenylphosphino)ferrocenedichloropalladium(II) (0.0587 g, 0.0713 mmol) in dioxane (8 mL) at 0° C. under N₂ was added dimethylzinc (0.713 mL, 1.43 mmol, 2 M solution in toluene). The reaction mixture was immediately heated to 100° C. for 30 minutes. The reaction mixture was cooled to 0° C. and quenched with MeOH (0.800 mL). The reaction mixture was diluted with EtOAc and washed with 1 M HCl. The aqueous layer was back extracted with EtOAc (1×). The combined organic layers were washed with saturated NaCl, dried (Na₂SO₄) and concentrated under reduced pressure to a dark yellow gum. Purification by flash column chromatography (methylene chloride/EtOAc, 15:1) gave 0.164 g (53%) pure desired product as a yellow crystalline solid.

Step E: Preparation of methyl-(2-fluoro-4-iodophenylamino)-1,5-dimethyl-6-oxo-1,6-dihydropyridine-3-carboxylate: To a solution of 2-fluoro-4-iodobenzenamine (0.058 g, 0.31 mmol) in THF (2 mL) at −78° C. under N₂ was added lithium bis(trimethylsilyl)amide (0.56 mL, 0.56 mmol, 1 M solution in hexanes) dropwise. The reaction mixture was stirred for one hour at −78° C. Methyl 2-chloro-1,5-dimethyl-6-oxo-1,6-dihydropyridine-3-carboxylate (0.060 g, 0.28 mmol) was then added dropwise as a solution in THF (1 mL) and the reaction mixture was stirred for 25 minutes at −78° C. The reaction mixture was quenched by the addition of H₂O and the pH was adjusted with 0.1M HCl and then diluted with EtOAc and saturated NaCl and the layers separated. The aqueous layer was back extracted with EtOAc (1×). The combined EtOAc layers were dried (Na₂SO₄) and concentrated under reduced pressure. Purification by flash column chromatography (methylene chloride/EtOAc, 20:1) gave 0.086 g (84%) pure desired product as a white crystalline solid. MS ESI (+) m/z 417 (M+1) detected; ¹H NMR (400 MHz, CDCl₃) δ 9.56 (s, 1H), 7.79 (s, 1H), 7.49 (d, 1H), 7.36 (d, 1H), 6.43 (t, 1H), 3.85 (s, 3H), 3.30 (s, 3H), 2.15 (s, 3H).

Step F: Preparation of 2-(2-fluoro-4-iodophenylamino)-1,5-dimethyl-6-oxo-N-(2-(vinyloxy)ethoxy)-1,6-dihydropyridine-3-carboxamide: To a solution of methyl 2-(2-fluoro-4-iodophenylamino)-1,5-dimethyl-6-oxo-1,6-dihydropyridine-3-carboxylate (0.500 g, 1.20 mmol) in THF (60 mL) was added O-(2-vinyloxy-ethyl)-hydroxylamine (0.149 g, 1.44 mmol). The solution was cooled to 0° C. and lithium bis(trimethylsilyl)amide (4.81 ml, 4.81 mmol) (1 M solution in hexanes) was added dropwise. The reaction mixture was warmed to room temperature. After stirring for 10 minutes the reaction mixture was quenched by the addition of 1 M HCl and partitioned between EtOAc and saturated NaCl. The layers were separated and the organic layer was dried (Na₂SO₄) and concentrated under reduced pressure to yield a crude yellow solid that was used without purification in the next step.

Step G: Preparation of 2-(2-fluoro-4-iodophenylamino)-N-(2-hydroxyethoxy)-1,5-dimethyl-6-oxo-1,6-dihydropyridine-3-carboxamide: To a solution of crude 2-(2-fluoro-4-iodophenylamino)-1,5-dimethyl-6-oxo-N-(2-(vinyloxy)ethoxy)-1,6-dihydropyridine-3-carboxamide (0.585 g, 1.20 mmol) in ethanol (10 mL) was added aqueous 2 M HCl (3 mL). The reaction mixture was stirred for 45 minutes at room temperature. The pH of the reaction mixture was adjusted to pH 7 with 1 M NaOH. The reaction mixture was diluted with EtOAc and H₂O. The organic layer was separated and washed with saturated NaCl. The combined aqueous layers were back extracted with EtOAc (1×). The combined organic layers were dried (Na₂SO₄) and concentrated under reduced pressure. Purification by silica gel flash column chromatography (methylene chloride/MeOH, 15:1) gave 2-(2-fluoro-4-iodophenylamino)-N-(2-hydroxyethoxy)-1,5-dimethyl-6-oxo-1,6-dihydropyridine-3-carboxamide (0.421 g; 76% over two steps) as a pale yellow solid. MS ESI (+) m/z 462 (M+1) pattern detected; ¹H NMR (400 MHz, CDCl₃) δ 9.77 (s, 1H), 8.50 (s, 1H), 7.47 (d, 1H), 7.36 (d, 1H), 6.43 (t, 1H), 4.04 (br s, 2H), 3.85 (br s, 1H), 3.74 (br s, 2H), 3.29 (s, 3H), 2.14 (s, 3H).

The following test may be used to demonstrate the activity of AZD2171 in combination with AZD6244 or MEK Inhibitor II.

### Calu-6 Human Lung Cancer Xenograft Model

Experiments were conducted in female athymic mice (Swiss nu/nu genotype, ≧6 weeks of age). Calu-6 human lung tumour xenografts were established in mice by injecting 1×10⁶ cells (1000 volume containing 50% Matrigel®) subcutaneously in the dorsal flank. Tumour volumes were assessed using bilateral Vernier calliper measurement at least twice weekly and calculated using the formula (length×width)×√(length×width)×(π/6), where length was taken to be the longest diameter across the tumour and width the corresponding perpendicular. Mice were randomised into 4 treatment groups (10 mice/group) when the mean tumour volume reached approximately 0.2 cm³. Following randomisation, mice were treated orally (p.o.) for 24 days with either drug vehicle, AZD2171 (1.5 mg/kg administered once daily), or with AZD6244 (3 mg/kg administered twice daily). Vehicle for AZD2171 (administered once daily) was 1% Polysorbate 80 and vehicle for AZD6244 (administered twice daily) was H.P.M.C. (0.5% w/v Methocel/0.1% w/v Tween 80). An additional group of animals received a combination of AZD2171 and AZD6244, using the same doses, schedule and duration as used for single agent treatment but with AZD2171 being administered 2 h after the first daily dose of AZD6244.

Tumour growth inhibition from the start of treatment was assessed by comparison of the differences in tumour volume between control and treated groups. The effects of combination treatment were assessed by comparing any effect on tumour growth in the group of animals receiving AZD2171 plus AZD6244 with tumour growth in the groups where animals received single agent therapy alone.

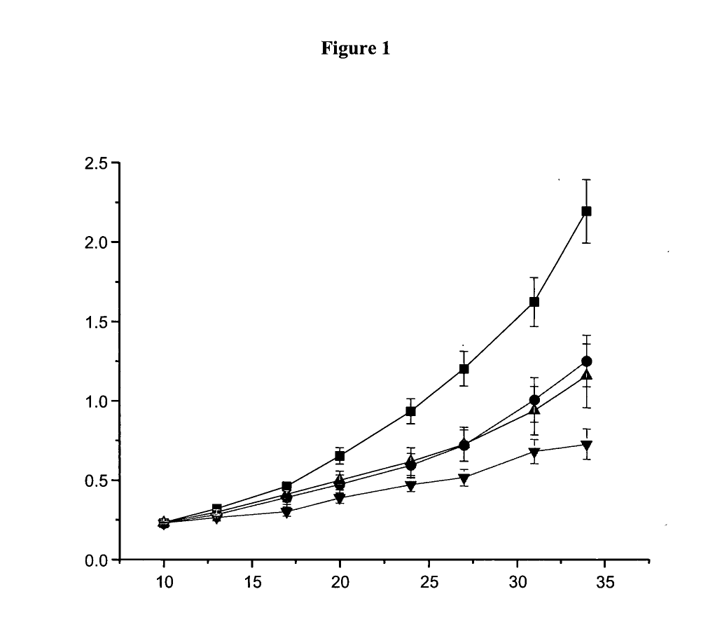

The data are shown graphically in FIG. 1 which shows the effect of AZD2171, AZD6244 or combination therapy on the growth of Calu-6 tumours in athymic mice wherein the x-axis is days post tumour inoculation and the y-axis is mean tumour volume in cm³±SEM. In the figure the squares relate to the vehicle control, the circles relate to AZD6244 (3 mg/kg twice daily), the upright triangles to AZD2171 (1.5 mg/kg once daily) and the inverted triangles to AZD6244 (3 mg/kg twice daily) and AZD2171 (1.5 mg/kg once daily) in combination.

The tumour growth inhibition of the combination was significantly greater than that achieved with AZD6244 alone (44% p=0.002) or AZD2171 alone (47% p=0.0015).

An analogous experiment may be used to look at the combination of AZD2171 and MEK Inhibitor II.

An analogous experiment may be used to look at the combinations of AZD2171 and AZD6244 or AZD2171 and MEK Inhibitor II with ionising radiation.

1-8. (canceled)

9. A pharmaceutical composition which comprises AZD2171 or a pharmaceutically acceptable salt thereof, and AZD6244 or a pharmaceutically acceptable salt thereof, in association with a pharmaceutically acceptable excipient or carrier.

10. A pharmaceutical composition which comprises AZD2171 or a pharmaceutically acceptable salt thereof, and MEK Inhibitor II or a pharmaceutically acceptable salt thereof, in association with a pharmaceutically acceptable excipient or carrier.

11. A kit comprising;
a) AZD2171 or a pharmaceutically acceptable salt thereof in a first unit dosage form;
b) AZD6244 or a pharmaceutically acceptable salt thereof in a second unit dosage form; and
c) container means for containing said first and second dosage forms.

12. A kit comprising;
a) AZD2171 or a pharmaceutically acceptable salt thereof in a first unit dosage form;
b) MEK Inhibitor II or a pharmaceutically acceptable salt thereof in a second unit dosage form; and
c) container means for containing said first and second dosage forms.

13-14. (canceled)

15. A method for the production of an antiangiogenic and/or vascular permeability reducing effect in a warm-blooded animal, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

16. A method for the treatment of a cancer in a warm-blooded animal, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof.

17. The method of claim 16 wherein said cancer is a cancer involving a solid tumour.

18. The method of claim 16 wherein said cancer is non-small cell lung cancer (NSCLC).

19. The method of claim 16 wherein said cancer is colorectal cancer (CRC).

20. The method of claim 16 wherein said cancer is cancer of the pancreas.

21. The method of claim 16 for wherein said cancer is malignant melanoma.

22. A method for the production of an antiangiogenic and/or vascular permeability reducing effect in a warm-blooded animal, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

23. A method for the treatment of a cancer in a warm-blooded animal, which comprises administering to said animal an effective amount of AZD2171 or a pharmaceutically acceptable salt thereof, before, after or simultaneously with an effective amount of either AZD6244 or a pharmaceutically acceptable salt thereof or MEK Inhibitor II or a pharmaceutically acceptable salt thereof, and before, after or simultaneously with an effective amount of ionising radiation.

24. The method according to claim 23 wherein said cancer is a cancer involving a solid tumour.

25. The method according to claim 23 wherein said cancer is non-small cell lung cancer (NSCLC).

26. The method according to claim 23 wherein said cancer is colorectal cancer (CRC).

27. The method according to claim 23 wherein said cancer is cancer of the pancreas.

28. The method according to claim 23 wherein said cancer is malignant melanoma.
